(19) United States
(12) Patent Application Publication
(10) Pub. No.: US 2012/0071340 A1
(43) Pub. Date: Mar. 22, 2012

(54) BIOMARKERS

(75) Inventors: Sabine Bahn, Cabridge (GB); Emanuel Schwarz, Cambridge (GB)

(73) Assignee: PSYNOVA NEUROTECH LTD., Cambridge (GB)

(21) Appl. No.: 13/203,822

(22) PCT Filed: Feb. 26, 2010
(86) PCT No.: PCT/GB10/50331

§ 371 Date: Dec. 2, 2011

(30) Foreign Application Priority Data
Feb. 27, 2009 (GB) 0903417.4

(51) Int. Cl. C40B 30/04; C40B 40/02; G01N 33/573; G01N 30/00; G01N 21/64; G01N 33/82; C12Q 1/70; H01J 49/26; C40B 40/10; G01N 33/566

(52) U.S. Cl. 506/9; 506/18; 506/14; 435/7.4; 436/501; 435/7.5; 435/7.92; 435/5; 250/282; 73/61.52

(57) ABSTRACT

The invention relates to a method of diagnosing or monitoring major depressive disorder.

### FIELD OF THE INVENTION

The invention relates to a method of diagnosing or monitoring major depressive disorder.

### BACKGROUND OF THE INVENTION

Major depressive disorder is a mental disorder characterized by a pervasive low mood, low self-esteem, and loss of interest or pleasure in normally enjoyable activities. The term “major depressive disorder” (which is also known as clinical depression, major depression, unipolar depression, or unipolar disorder) was selected by the American Psychiatric Association for this symptom cluster under mood disorders in the 1980 version of the Diagnostic and Statistical Manual of Mental Disorders (DSM-III) classification, and has become widely used since.

The general term depression is often used to describe the disorder, but as it is also used to describe a depressed mood, more precise terminology is preferred in clinical and research use. Major depression is a disabling condition which adversely affects a person's family, work or school life, sleeping and eating habits, and general health. In the United States, approximately 3.4% of people with major depression commit suicide, and up to 60% of all people who commit suicide have depression or another mood disorder.

The diagnosis of major depressive disorder is based on the patient's self-reported experiences, behaviour reported by relatives or friends, and a mental status exam. There is no laboratory test for major depression, although physicians generally request tests for physical conditions that may cause similar symptoms. The most common time of onset is between the ages of 30 and 40 years, with a later peak between 50 and 60 years. Major depression is reported about twice as frequently in women as in men, although men are at higher risk for suicide.

Most patients are treated in the community with antidepressant medication and some with psychotherapy or counseling. Hospitalization may be necessary in cases with associated self-neglect or a significant risk of harm to self or others. A minority are treated with electroconvulsive therapy (ECT), under a short-acting general anaesthetic.

The course of the disorder varies widely, from one episode lasting months to a lifelong disorder with recurrent major depressive episodes. Depressed individuals have shorter life expectancies than those without depression, in part because of greater susceptibility to medical illnesses. Current and former patients may be stigmatized.

The understanding of the nature and causes of depression has evolved over the centuries, though many aspects of depression remain incompletely understood and are the subject of discussion and research.

### SUMMARY OF THE INVENTION

According to a first aspect of the invention, there is provided the use of IL-17, IgA, Cortisol (CORT), Apolipoprotein A1, IL-6, Complement 3 (C3), Factor VII, Serum Amyloid P (SAP or APCS), Beta 2 Microglobulin, ICAM-1, IL-1 beta, TNF alpha, MIF, Angiotensinogen, NrCAM (Neuronal cell adhesion molecule), CD40, Cancer Antigen 125 (CA125), HCC 4 (CCL6; SCYA6), Eotaxin 3 (CCL26 or SCYA26), VEGF, Haptoglobin (HP), IL-1 alpha, Apolipoprotein H (Beta-2 Glycoprotein) and TIMP 1 as a specific panel of analyte biomarkers for major depressive disorder, or predisposition thereto

According to a second aspect of the invention, there is provided a method of diagnosing or monitoring major depressive disorder, or predisposition thereto, comprising detecting and/or quantifying, in a sample from a test subject, the analyte biomarkers defined herein.

According to a third aspect of the invention, there is provided a method of diagnosing major depressive disorder, or predisposition in an individual thereto, comprising:

(a) obtaining a biological sample from an individual;

(b) quantifying the amounts of the analyte biomarkers as defined herein;

(c) comparing the amounts of the analyte biomarkers in the biological sample with the amounts present in a normal control biological sample from a normal subject, such that a difference in the level of the analyte biomarkers in the biological sample is indicative of major depressive disorder, or predisposition thereto.

According to a fourth aspect of the invention, there is provided a method of io monitoring efficacy of a therapy in a subject having, suspected of having, or of being predisposed to major depressive disorder, comprising detecting and/or quantifying, in a sample from said subject, one or more of the first peptide biomarkers defined herein.

According to a fifth aspect of the invention, there is provided a method of determining the efficacy of therapy for major depressive disorder in an individual subject comprising:

(a) obtaining a biological sample from an individual;

(b) quantifying the amounts of the analyte biomarkers as defined herein;

(c) comparing the amounts of the analyte biomarkers in the biological sample with the amounts present in a sample obtained from the individual on a previous occasion, such that a difference in the level of the analyte biomarkers in the biological sample is indicative of a beneficial effect of the therapy.

According to a sixth aspect of the invention, there is provided a method of monitoring efficacy of a therapy in a subject having, suspected of having, or of being predisposed to major depressive disorder, comprising detecting and/or quantifying, in a sample from said subject, two or more of the second peptide biomarkers defined herein.

A further aspect of the invention provides ligands, such as naturally occurring or chemically synthesised compounds, capable of specific binding to the peptide biomarker. A ligand according to the invention may comprise a peptide, an antibody or a fragment thereof, or an aptamer or oligonucleotide, capable of specific binding to the peptide biomarker. The antibody can be a monoclonal antibody or a fragment thereof capable of specific binding to the peptide biomarker. A ligand according to the invention may be labelled with a detectable marker, such as a luminescent, fluorescent or radioactive marker; alternatively or additionally a ligand according to the invention may be labelled with an affinity tag, e.g. a biotin, avidin, streptavidin or His (e.g. hexa-His) tag.

A biosensor according to the invention may comprise the peptide biomarker or a io structural/shape mimic thereof capable of specific binding to an antibody against the peptide biomarker. Also provided is an array comprising a ligand or mimic as described herein.

Also provided by the invention is the use of one or more ligands as described herein, which may be naturally occurring or chemically synthesised, and is suitably a peptide, antibody or fragment thereof, aptamer or oligonucleotide, or the use of a biosensor of the invention, or an array of the invention, or a kit of the invention to detect and/or quantify the peptide. In these uses, the detection and/or quantification can be performed on a biological sample such as from the group consisting of CSF, whole blood, blood serum, plasma, urine, saliva, or other bodily fluid, breath, e.g. as condensed breath, or an extract or purification therefrom, or dilution thereof.

Diagnostic or monitoring kits are provided for performing methods of the invention. Such kits will suitably comprise a ligand according to the invention, for detection and/or quantification of the peptide biomarker, and/or a biosensor, and/or an array as described herein, optionally together with instructions for use of the kit.

A further aspect of the invention is a kit for monitoring or diagnosing major depressive disorder, comprising a biosensor capable of detecting and/or quantifying one or more of the first peptide biomarkers as defined herein.

A further aspect of the invention is a kit for monitoring or diagnosing major depressive disorder, comprising a biosensor capable of detecting and/or quantifying two or more of the second peptide biomarkers as defined herein.

Biomarkers for major depressive disorder are essential targets for discovery of novel targets and drug molecules that retard or halt progression of the disorder. As the level of the peptide biomarker is indicative of disorder and of drug response, the biomarker is useful for identification of novel therapeutic compounds in in vitro and/or in vivo assays. Biomarkers of the invention can be employed in methods for screening for compounds that modulate the activity of the peptide.

Thus, in a further aspect of the invention, there is provided the use of a ligand, as described, which can be a peptide, antibody or fragment thereof or aptamer or oligonucleotide according to the invention; or the use of a biosensor according to the invention, or an array according to the invention; or a kit according to the invention, to identify a substance capable of promoting and/or of suppressing the generation of the biomarker.

Also there is provided a method of identifying a substance capable of promoting or suppressing the generation of the peptide in a subject, comprising administering a test substance to a subject animal and detecting and/or quantifying the level of the peptide biomarker present in a test sample from the subject.

### DETAILED DESCRIPTION OF THE INVENTION

According to a first aspect of the invention, there is provided the use of IL-17, IgA, Cortisol (CORT), Apolipoprotein A1, IL-6, Complement 3 (C3), Factor VII, Serum Amyloid P (SAP or APCS), Beta 2 Microglobulin, ICAM-1, IL-1 beta, TNF alpha, MIF, Angiotensinogen, NrCAM (Neuronal cell adhesion molecule), CD40, Cancer Antigen 125 (CA125), HCC 4 (CCL6; SCYA6), Eotaxin 3 (CCL26 or SCYA26), VEGF, Haptoglobin (HP), IL-1 alpha, Apolipoprotein H (Beta-2 Glycoprotein) and TIMP 1 as a specific panel of analyte biomarkers for major depressive disorder, or predisposition thereto.

The invention provides a panel of analyte biomarkers for the effective and sensitive diagnosis of major depressive disorder. The panel according to the first aspect of the invention was identified by selection according to particular parameters following the results of Study 1. For example, controls were compared from centre 1 against controls from centre 2. 111 proteins were found to be sign different (i.e. p<0.05, t-test, two-tailed). All markers among these 111 which were sign altered between MDD and controls were removed. This resulted in 34 markers. ANCOVA analysis was then performed using age and gender as covariates which reduced the selected markers from 34 to 30. Of the 30 remaining markers, 5 were autoimmune antibodies and 1 marker (Peptide YY) was only detected in 11% of the samples. This detailed filtering system therefore resulted in the identification of the specific panel of 24 analyte biomarkers of the first aspect of the invention.

In one embodiment, the panel according to the first aspect of the invention IL-17, Cortisol (CORT), IL-6, Complement 3 (C3), Factor VII, Serum Amyloid P (SAP or APCS), Beta 2 Microglobulin, ICAM-1, IL-1 beta, TNF alpha, MIF, CD40, Cancer Antigen 125 (CA125), HCC 4 (CCL6; SCYA6), Eotaxin 3 (CCL26 or SCYA26), VEGF, Haptoglobin (HP), Apolipoprotein H (Beta-2 Glycoprotein) and TIMP 1. This sub-set panel of the first aspect of the invention provides a set of analyte biomarkers wherein levels were found to be increased in patients with major depressive disorder in accordance with the study presented herein.

In an alternative embodiment, the panel according to the first aspect of the invention comprises IgA, Apolipoprotein A1, Angiotensinogen, NrCAM and IL-1 alpha. This sub-set panel of the first aspect of the invention provides a set of analyte biomarkers wherein levels were found to be decreased in patients with major depressive disorder in accordance with the study presented herein.

In one embodiment, the panel according to the first aspect of the invention additionally comprises one or more analyte biomarkers selected from: Creatine Kinase MB (CK-MB), ENA 78 (CXCL5), Endothelin 1, FABP (Fatty acid binding protein), MDC (CCL22), MIP 1 beta, PARC (p53-associated parkin-like cytoplasmic protein), Peptide YY (PYY), Prostatic Acid Phosphatase, Sortilin (SORT), Stem Cell Factor (SCF), T3 Antibody, Thrombopoietin (THPO), TSP 1 (thrombospondin-1), Scl 70 Antibody, Histone H2B Antibody, Histone H1 Antibody, Histone Antibody, PM 1 Antibody, Histone H3 Antibody, Histone H2a Antibody, Anti Nuclear Antibody, SSB Antibody, Centromere Protein B Antibody, Rubeola, Hepatitis C Core, Hepatitis E Virus orf 3.3KD, Smith Antibody, HSP 32 HO Antibody, Parainfluenza 1, Hepatitis D, Proteinase 3 cANCA Antibody, HSP 71 Antibody, Collagen Type 2 Antibody, Mycoplasma pneumoniae (M. pneumoniae), Trypanosoma cruzi (T. cruzi), Hepatitis A, RNP Antibody, Hepatitis C NS4, RNP (a) Antibody, HIV 1 gp120, Chlamydia trachomatis (C. trachomatis), Helicobacter pylori (H. pylori), Mumps, Bordetella pertussis (B. pertussi), Beta-2 Glycoprotein Antibody (B2GP), Hepatitis C NS3, Collagen Type 4 Antibody (COL4), Poliovirus, Hepatitis C NS5, CTGF (Connective Tissue Growth Factor), Ferritin (FTL), Fibrinogen (FGA), G-CSF, IL-12 p70, IL-13, IL-15, IL-16, IL-18, IL-1 ra, IL-4, IL-5, IL-7, IL-8, Leptin, MIP-1 alpha, PDGF (Platelet-derived growth factor), SOD, Ribosomal P Antibody, HSC 70 Antibody, HSP90 alpha Antibody, HSP90 beta Antibody and Varicella zoster (V. zoster; VZV). The analyte biomarkers of this embodiment of the invention were surprisingly found to be significantly altered in Study 1 conducted herein.

In one embodiment, the panel according to the first aspect of the invention additionally comprises one or more analyte biomarkers selected from: Alpha-Fetoprotein, Glutathione S-Transferase-α, Eotaxin, Toxoplasma, IGF-BP2 and Brain-Derived Neurotrophic Factor.

In one embodiment, the panel according to the first aspect of the invention additionally comprises one or more analyte biomarkers selected from: Alpha-Fetoprotein, SOD, Glutathione S-Transferase-α, IL-15, Eotaxin, Toxoplasma, IGF-BP2 and Brain-Derived Neurotrophic Factor. The analyte biomarkers of this embodiment of the invention were surprisingly found to be significantly altered in Study 2 conducted herein.

In a further embodiment, the one or more analyte biomarkers are selected from SOD and IL-15. The analyte biomarkers of this embodiment of the invention were surprisingly found to be significantly altered in both studies conducted herein.

In one embodiment of the panel according to the first aspect of the invention, the one or more analyte biomarkers are selected from Alpha-Fetoprotein, Bordetella pertussis (B. pertussi), Hepatitis C NS5 and Beta-2 Glycoprotein Antibody (B2GP).

According to a further aspect of the invention, there is provided the use of IL-17, IgA, Cortisol (CORT), Apolipoprotein A1, IL-6, Complement 3 (C3), Factor VII, Serum Amyloid P (SAP or APCS), Beta 2 Microglobulin, ICAM-1, IL-1 beta, TNF alpha, MIF, Angiotensinogen, NrCAM (Neuronal cell adhesion molecule), CD40, Cancer Antigen 125 (CA125), HCC 4 (CCL6; SCYA6), Eotaxin 3 (CCL26 or SCYA26), VEGF, Haptoglobin (HP), IL-1 alpha, Apolipoprotein H (Beta-2 Glycoprotein), TIMP 1, Creatine Kinase MB (CK-MB), ENA 78 (CXCL5), Endothelin 1, FABP (Fatty acid binding protein), MDC (CCL22), MIP 1 beta, PARC (p53-associated parkin-like cytoplasmic protein), Peptide YY (PYY), Prostatic Acid Phosphatase, Sortilin (SORT), Stem Cell Factor (SCF), T3 Antibody, Thrombopoietin (THPO), TSP 1 (thrombospondin-1), Scl 70 Antibody, Histone H2B Antibody, Histone H1 Antibody, Histone Antibody, PM 1 Antibody, Histone H3 Antibody, Histone H2a Antibody, Anti Nuclear Antibody, SSB Antibody, Centromere Protein B Antibody, Rubeola, Hepatitis C Core, Hepatitis E Virus orf 3.3KD, Smith Antibody, HSP 32 HO Antibody, Parainfluenza 1, Hepatitis D, Proteinase 3 cANCA Antibody, HSP 71 Antibody, Collagen Type 2 Antibody, Mycoplasma pneumoniae (M. pneumoniae), Trypanosoma cruzi (T. cruzi), Hepatitis A, RNP Antibody, Hepatitis C NS4, RNP (a) Antibody, HIV 1 gp120, Chlamydia trachomatis (C. trachomatis), Helicobacter pylori (H. pylori), Mumps, Bordetella pertussis (B. pertussi), Beta-2 Glycoprotein Antibody (B2GP), Hepatitis C NS3, Collagen Type 4 Antibody (COL4), Poliovirus, Hepatitis C NS5, CTGF (Connective Tissue Growth Factor), Ferritin (FTL), Fibrinogen (FGA), G-CSF, IL-12 p70, IL-13, IL-15, IL-16, IL-18, IL-1 ra, IL-4, IL-5, IL-7, IL-8, Leptin, MIP-1 alpha, PDGF (Platelet-derived growth factor), SOD, Ribosomal P Antibody, HSC 70 Antibody, HSP90 alpha Antibody, HSP90 beta Antibody, Varicella zoster (V. zoster; VZV), Alpha-Fetoprotein, Glutathione S-Transferase-α, Eotaxin, Toxoplasma, IGF-BP2 and Brain-Derived Neurotrophic Factor as a specific panel of analyte biomarkers for major depressive disorder, or predisposition thereto.

The term “biomarker” means a distinctive biological or biologically derived indicator of a process, event, or condition. Peptide biomarkers can be used in methods of diagnosis, e.g. clinical screening, and prognosis assessment and in monitoring the results of therapy, identifying patients most likely to respond to a particular therapeutic treatment, drug screening and development. Biomarkers and uses thereof are valuable for identification of new drug treatments and for discovery of new targets for drug treatment.

It will be readily apparent to the skilled person that the first and second peptides listed herein are known and have been described in the literature, however, for completeness, full characterising information for these peptides is provided in Table 1:

TABLE 1
Characterising Information of the First and Second
Peptides of the Invention

| Analyte | Accession Number |
|---|---|
| Angiotensinogen | P01019 |
| Alpha-Fetoprotein | |
| Anti Nuclear Antibody | |
| Apolipoprotein A1 | P02647 |
| Apolipoprotein H | P02749 |
| B. pertussis | |
| Beta 2 Glycoprotein Antibody | |
| Beta 2 Microglobulin | P61769 |
| Brain-Derived Neurotrophic | |
| Factor | |
| C. trachomatis | |
| Cancer Antigen 125 | Q14596 |
| CD40 | P25942 |
| Centromere Protein B Antibody | |
| Collagen Type 2 Antibody | |
| Collagen Type 4 Antibody | |
| Complement 3 | P01026 |
| Cortisol | |
| Creatine Kinase MB | P06732 |
| CTGF (Connective Tissue Growth Factor) | P29279 |
| ENA 78 | P42830 |
| Endothelin 1 | P05305 |
| Eotaxin | |
| Eotaxin 3 | P05305 |
| FABP | P07148 |
| Factor VII | P08709 |
| Ferritin | P02794 |
| Fibrinogen | P02679 |
| G-CSF | P09919 |
| Glutathione S-Transferase-α | |
| H. pylori | |
| Haptoglobin | P00738 |
| HCC 4 | O15467 |
| Hepatitis A | |
| Hepatitis C Core | |
| Hepatitis C NS3 | |
| Hepatitis C NS4 | |
| Hepatitis D | |
| Hepatitis E Virus orf 3.3KD | |
| Histone Antibody | |
| Histone H1 Antibody | |
| Histone H2a Antibody | |
| Histone H2b Antibody | |
| Histone H3 Antibody | |
| HIV-1 gp120 | |
| HSC 70 Antibody | |
| HSP 32 HO Antibody | |
| HSP 71 Antibody | |
| HSP 90 alpha Antibody | |
| HSP 90 beta Antibody | |
| ICAM-1 | P05362 |
| IgA | P01876 |
| IGF BP-2 | |
| IL-12 p70 | |
| IL-13 | P35225 |
| IL-15 | P40933 |
| IL-16 | Q14005 |
| IL-17 | Q16552 |
| IL-18 | Q14116 |
| IL-1 alpha | P01583 |
| IL-1 beta | P01584 |
| IL-1 ra | P18510 |
| IL-4 | P05112 |
| IL-5 | P05113 |
| IL-6 | P05231 |
| IL-7 | P13232 |
| IL-8 | P10145 |
| Leptin | P41159 |
| M. pneumoniae | |
| MDC | Q14676 |
| MIF | P14174 |
| MIP-1 alpha | P10147 |
| MIP-1 beta | P10147 |
| Mumps | |
| NrCAM | Q92823 |
| Parainfluenza 1 | |
| PARC | P55774 |
| PDGF | P01127 |
| Peptide YY | P10082 |
| PM-1 Antibody | |
| Polio Virus | |
| Prostatic Acid Phosphatase | P15309 |
| Proteinase 3 cANCA Antibody | |
| Ribosomal P Antibody | |
| RNP a Antibody | |
| RNP Antibody | |
| Rubeola | |
| Scl 70 Antibody | |
| Serum Amyloid P | |
| Smith Antibody | |
| SOD | P08294 |
| Sortilin | Q99523 |
| SSB Antibody | |
| Stem Cell Factor | P21583 |
| T. cruzi | |
| T3 Antibody | |
| Thrombopoietin | P40225 |
| TIMP-1 | P01033 |
| TNF alpha | P01375 |
| Toxoplasma | |
| TSP.1 | P07996 |
| V. zoster | |
| VEGF | P15692 |

According to one particular aspect of the invention, there is provided the use of one or more first peptides selected from: Angiotensinogen, Apolipoprotein H (Beta-2 Glycoprotein), Cancer Antigen 125 (CA125), Creatine Kinase MB (CK-MB), ENA 78 (CXCL5), Endothelin 1, FABP (Fatty acid binding protein), Factor VII, MDC (CCL22), MIP 1 beta, PARC (p53-associated parkin-like cytoplasmic protein), Peptide YY (PYY), Prostatic Acid Phosphatase, Serum Amyloid P (SAP or APCS), Sortilin (SORT), Stem Cell Factor (SCF), T3 Antibody, Thrombopoietin (THPO), TSP 1 (thrombospondin-1), Scl 70 Antibody, Histone H2B Antibody, Histone H1 Antibody, Histone Antibody, PM 1 Antibody, Histone H3 Antibody, Histone H2a Antibody, Anti Nuclear Antibody, SSB Antibody, Centromere Protein B Antibody, Rubeola, Hepatitis C Core, Hepatitis E Virus orf 3.3KD, Smith Antibody, HSP 32 HO Antibody, Parainfluenza 1, Hepatitis D, Proteinase 3 cANCA Antibody, HSP 71 Antibody, Collagen Type 2 Antibody, Mycoplasma pneumoniae (M. pneumoniae), Trypanosoma cruzi (T. cruzi), Hepatitis A, RNP Antibody, HCC 4 (CCL6; SCYA6), Hepatitis C NS4, RNP (a) Antibody, HIV 1 gp120, Chlamydia trachomatis (C. trachomatis), Helicobacter pylori (H. pylori), Mumps, Bordetella pertussis (B. pertussi), Beta-2 Glycoprotein Antibody (B2GP), Hepatitis C NS3, Collagen Type 4 Antibody (COL4), Poliovirus, Hepatitis C NS5, as a biomarker for major depressive disorder, or predisposition thereto.

According to a further particular aspect of the invention, there is provided the use of one or more first peptides selected from: Alpha-Fetoprotein, Angiotensinogen, Apolipoprotein H (Beta-2 Glycoprotein), Cancer Antigen 125 (CA125), Creatine Kinase MB (CK-MB), ENA 78 (CXCL5), Endothelin 1, MDC (CCL22), IGF-BP2, MIP 1 beta, PARC (p53-associated parkin-like cytoplasmic protein), Peptide YY (PYY), Prostatic Acid Phosphatase, Serum Amyloid P (SAP or APCS), Sortilin (SORT), Stem Cell Factor (SCF), T3 Antibody, Thrombopoietin (THPO), TSP 1 (thrombospondin-1), Scl 70 Antibody, Histone H2B Antibody, Histone Antibody, PM 1 Antibody, Histone H3 Antibody, Histone H2a Antibody, Anti Nuclear Antibody, SSB Antibody, Centromere Protein B Antibody, Rubeola, Hepatitis C Core, Hepatitis E Virus orf 3.3KD, Smith Antibody, HSP 32 HO Antibody, Parainfluenza 1, Hepatitis D, Proteinase 3 cANCA Antibody, HSP 71 Antibody, Collagen Type 2 Antibody, Mycoplasma pneumoniae (M. pneumoniae), Trypanosoma cruzi (T. cruzi), Hepatitis A, RNP Antibody, HCC 4 (CCL6; SCYA6), Hepatitis C NS4, RNP (a) Antibody, HIV 1 gp120, Chlamydia trachomatis (C. trachomatis), Helicobacter pylori (H. pylori), Mumps, Bordetella pertussis (B. pertussi), Beta-2 Glycoprotein Antibody (B2GP), Hepatitis C NS3, Collagen Type 4 Antibody (COL4), Poliovirus, Hepatitis C NS5, as a biomarker for major depressive disorder, or predisposition thereto.

In one embodiment, the one or more first peptides are selected from: Scl 70 Antibody, Histone H2B Antibody, Histone Antibody, PM 1 Antibody, Histone H3 Antibody, Histone H2a Antibody, Anti Nuclear Antibody, SSB Antibody, Centromere Protein B Antibody, Rubeola, Hepatitis C Core, Hepatitis E Virus orf 3.3KD, Smith Antibody, HSP 32 HO Antibody, Parainfluenza 1, Hepatitis D, Proteinase 3 cANCA Antibody, HSP 71 Antibody, Collagen Type 2 Antibody, Mycoplasma pneumoniae (M. pneumoniae), Trypanosoma cruzi (T. cruzi), Hepatitis A, RNP Antibody, HCC 4 (CCL6; SCYA6), Hepatitis C NS4, RNP (a) Antibody, HIV 1 gp120, Chlamydia trachomatis (C. trachomatis), Helicobacter pylori (H. pylori), Mumps, Bordetella pertussis (B. pertussi), Beta-2 Glycoprotein Antibody (B2GP), Hepatitis C NS3, Collagen Type 4 Antibody (COL4), Poliovirus and Hepatitis C NS5.

In one embodiment of any of the previously mentioned aspects of the invention, the first peptide is other than Creatine Kinase MB (CK-MB). In one embodiment of any of the previously mentioned aspects of the invention, the first peptide is other than MIP-1 beta. In one embodiment of any of the previously mentioned aspects of the invention, the first peptide is other than Serum Amyloid P (SAP or APCS). In one embodiment of any of the previously mentioned aspects of the invention, the first peptide is other than Collagen Type 2 Antibody. In one embodiment of any of the previously mentioned aspects of the invention, the first peptide is other than Collagen Type 4 Antibody (COL4). In one embodiment of any of the previously mentioned aspects of the invention, the first peptide is other than Apolipoprotein H (Beta-2 Glycoprotein).

In one embodiment of any of the previously mentioned aspects of the invention, the first peptide is selected from: Angiotensinogen, Cancer Antigen 125 (CA125), ENA 78 (CXCL5), Endothelin 1, MDC (CCL22), PARC (p53-associated parkin-like cytoplasmic protein), Peptide YY (PYY), Prostatic Acid Phosphatase, Sortilin (SORT), Stem Cell Factor (SCF), T3 Antibody, Thrombopoietin (THPO), TSP 1 (thrombospondin-1), Scl 70 Antibody, Histone H2B Antibody, Histone Antibody, PM 1 Antibody, Histone H3 Antibody, Histone H2a Antibody, Anti Nuclear Antibody, SSB Antibody, Centromere Protein B Antibody, Rubeola, Hepatitis C Core, Hepatitis E Virus orf 3.3KD, Smith Antibody, HSP 32 HO Antibody, Parainfluenza 1, Hepatitis D, Proteinase 3 cANCA Antibody, HSP 71 Antibody, Mycoplasma pneumoniae (M. pneumoniae), Trypanosoma cruzi (T. cruzi), Hepatitis A, RNP Antibody, HCC 4 (CCL6; SCYA6), Hepatitis C NS4, RNP (a) Antibody, HIV 1 gp120, Chlamydia trachomatis (C. trachomatis), Helicobacter pylori (H. pylori), Mumps, Bordetella pertussis (B. pertussi), Beta-2 Glycoprotein Antibody (B2GP), Hepatitis C NS3, Poliovirus and Hepatitis C NS5.

According to a further particular aspect of the invention, there is provided the use of two or more second peptides selected from: Apolipoprotein A1, Beta 2 Microglobulin, CD40, Complement 3 (C3), Cortisol (CORT), CTGF (Connective Tissue Growth Factor), Eotaxin 3 (CCL26 or SCYA26), Ferritin (FTL), Fibrinogen (FGA), G-CSF, Haptoglobin (HP), ICAM-1, IgA, IL-12 p70, IL-13, IL-15, IL-16, IL-17, IL-18, IL-1 alpha, IL-1 beta, IL-1 ra, IL-4, IL-5, IL-6, IL-7, IL-8, Leptin, MIF, MIP-1 alpha, NrCAM (Neuronal cell adhesion molecule), PDGF (Platelet-derived growth factor), SOD, TIMP-1, TNF alpha, VEGF, Ribosomal P Antibody, HSC 70 Antibody, HSP90 alpha Antibody, HSP90 beta Antibody, Varicella zoster (V. zoster; VZV), as a biomarker for major depressive disorder, or predisposition thereto.

According to a yet further particular aspect of the invention, there is provided the use of two or more second peptides selected from: Apolipoprotein A1, Beta 2 Microglobulin, Brain-Derived Neurotrophic Factor, CD40, Complement 3 (C3), Cortisol (CORT), CTGF (Connective Tissue Growth Factor), Eotaxin, Eotaxin 3 (CCL26 or SCYA26), Fatty Acid Binding Protein (FABP), Factor VII, Ferritin (FTL), Fibrinogen (FGA), G-CSF, Glutathione S-Transferase-α, Haptoglobin (HP), Histone H1 Antibody, ICAM-1, IgA, IL-12 p70, IL-13, IL-15, IL-16, IL-17, IL-18, IL-1 alpha, IL-1 beta, IL-1 ra, IL-4, IL-5, IL-6, IL-7, IL-8, Leptin, MIF, MIP-1 alpha, NrCAM (Neuronal cell adhesion molecule), PDGF (Platelet-derived growth factor), SOD, TIMP-1, TNF alpha, Toxoplasma, VEGF, Ribosomal P Antibody, HSC 70 Antibody, HSP90 alpha Antibody, HSP90 beta Antibody, Varicella zoster (V. zoster; VZV), as a biomarker for major depressive disorder, or predisposition thereto.

According to a further aspect of the invention, there is provided the use of two or more second peptides selected from: Ribosomal P Antibody, HSC 70 Antibody, HSP90 alpha Antibody, HSP90 beta Antibody, Varicella zoster (V. zoster; VZV), as a biomarker for major depressive disorder, or predisposition thereto.

In one embodiment of any of the previously mentioned aspects of the invention, the one or more second peptides additionally comprise Creatine Kinase MB (CK-MB). In one embodiment of any of the previously mentioned aspects of the invention, the one or more second peptides additionally comprise MIP-1 beta. In one embodiment of any of the previously mentioned aspects of the invention, the one or more second peptides additionally comprise Serum Amyloid P (SAP or APCS). In one embodiment of any of the previously mentioned aspects of the invention, the one or more second peptides additionally comprise Collagen Type 2 Antibody. In one embodiment of any of the previously mentioned aspects of the invention, the one or more second peptides additionally comprise Collagen Type 4 Antibody (COL4). In one embodiment of any of the previously mentioned aspects of the invention, the one or more second peptides additionally comprise Apolipoprotein H (Beta-2 Glycoprotein).

According to a further aspect of the invention, there is provided the use of two or more second peptides selected from: Apolipoprotein A1, Apolipoprotein H (Beta-2 Glycoprotein), Beta 2 Microglobulin, CD40, Complement 3 (C3), Cortisol (CORT), Creatine Kinase MB (CK-MB), CTGF (Connective Tissue Growth Factor), Eotaxin 3 (CCL26 or SCYA26), Fatty Acid Binding Protein (FABP), Factor VII, Ferritin (FTL), Fibrinogen (FGA), G-CSF, Haptoglobin (HP), Histone H1 Antibody, ICAM-1, IgA, IL-12 p70, IL-13, IL-15, IL-16, IL-17, IL-18, IL-1 alpha, IL-1 beta, IL-1 ra, IL-4, IL-5, IL-6, IL-7, IL-8, Leptin, MIF, MIP-1 alpha, MIP-1 beta, NrCAM (Neuronal cell adhesion molecule), PDGF (Platelet-derived growth factor), Serum Amyloid P (SAP or APCS), SOD, TIMP-1, TNF alpha, VEGF, Ribosomal P Antibody, HSC 70 Antibody, HSP90 alpha Antibody, HSP90 beta Antibody, Collagen Type 2 Antibody, Varicella zoster (V. zoster; VZV), Collagen Type 4 Antibody (COL4), as a biomarker for major depressive disorder, or predisposition thereto.

According to a further aspect of the invention, there is provided the use of one or more peptides listed in Table 3, as a biomarker for major depressive disorder, or predisposition thereto. In particular, it can be noted that the biomarkers with a fold change of <1 are those wherein levels are decreased in patients with major depressive disorder. By contrast, the biomarkers with a fold change of >1 are those wherein levels are increased in patients with major depressive disorder.

For example, it can be noted that the levels of the following biomarkers decreased in patients with major depressive disorder: IL-5, IgA, Apolipoprotein A1, TSP 1, Peptide YY, Creatine Kinase MB, Angiotensinogen, NrCAM, Sortilin, Endothelin 1, IL-1 alpha, II-13 and CTGF (Connective Tissue Growth Factor).

Furthermore, it can be noted that the levels of the following biomarkers increased in patients with major depressive disorder: IL-17, Cortisol (CORT), IL-6, Complement 3 (C3), Factor VII, Serum Amyloid P (SAP or APCS), Beta 2 Microglobulin, ICAM-1, IL-1 beta, TNF alpha, MIF, CD40, Cancer Antigen 125 (CA125), HCC 4 (CCL6; SCYA6), Eotaxin 3 (CCL26 or SCYA26), VEGF, Haptoglobin (HP), Apolipoprotein H (Beta-2 Glycoprotein), TIMP 1, ENA 78 (CXCL5), FABP (Fatty acid binding protein), MDC (CCL22), MIP 1 beta, PARC (p53-associated parkin-like cytoplasmic protein), Prostatic Acid Phosphatase, Stem Cell Factor (SCF), T3 Antibody, Thrombopoietin (THPO), Scl 70 Antibody, Histone H2B Antibody, Histone H1 Antibody, Histone Antibody, PM 1 Antibody, Histone H3 Antibody, Histone H2a Antibody, Anti Nuclear Antibody, SSB Antibody, Centromere Protein B Antibody, Rubeola, Hepatitis C Core, Hepatitis E Virus orf 3.3KD, Smith Antibody, HSP 32 HO Antibody, Parainfluenza 1, Hepatitis D, Proteinase 3 cANCA Antibody, HSP 71 Antibody, Collagen Type 2 Antibody, Mycoplasma pneumoniae (M. pneumoniae), Trypanosoma cruzi (T. cruzi), Hepatitis A, RNP Antibody, Hepatitis C NS4, RNP (a) Antibody, HIV 1 gp120, Chlamydia trachomatis (C. trachomatis), Helicobacter pylori (H. pylori), Mumps, Bordetella pertussis (B. pertussi), Beta-2 Glycoprotein Antibody (B2GP), Hepatitis C NS3, Collagen Type 4 Antibody (COL4), Poliovirus, Hepatitis C NS5, Ferritin (FTL), Fibrinogen (FGA), G-CSF, IL-12 p70, IL-15, IL-16, IL-18, IL-1 ra, IL-4, IL-7, IL-8, Leptin, MIP-1 alpha, PDGF (Platelet-derived growth factor), SOD, Ribosomal P Antibody, HSC 70 Antibody, HSP90 alpha Antibody, HSP90 beta Antibody and Varicella zoster (V. zoster; VZV).

According to a further aspect of the invention, there is provided the use of IL-5, IgA, Apolipoprotein A1, TSP 1, Peptide YY, Creatine Kinase MB, Angiotensinogen, NrCAM, Sortilin, Endothelin 1, IL-1 alpha, Il-13 and CTGF (Connective Tissue Growth Factor) as a specific panel of analyte biomarkers for major depressive disorder, or predisposition thereto.

According to a further aspect of the invention, there is provided a method of diagnosing major depressive disorder, or predisposition thereto, in an individual thereto comprising
a) obtaining a biological sample from an individual;
b) quantifying the amounts of a panel of analyte biomarkers in the biological sample, wherein the panel of analyte biomarkers comprises IL-5, IgA, Apolipoprotein A1, TSP 1, Peptide YY, Creatine Kinase MB, Angiotensinogen, NrCAM, Sortilin, Endothelin 1, IL-1 alpha, Il -13 and CTGF (Connective Tissue Growth Factor); and
c) comparing the amounts of the panel of analyte biomarkers in the biological sample with the amounts present in a normal control biological sample from a normal subject, wherein a lower level of the panel of analyte biomarkers in the biological sample is indicative of major depressive disorder, or predisposition thereto.

In one embodiment, the lower level is a <1 fold difference relative to the control sample, such as a fold difference of 0.9, 0.8, 0.7, 0.6, 0.5, 0.4, 0.3, 0.2, 0.1, 0.05, 0.01 or any ranges therebetween. In one embodiment, the lower level is between a 0.1 and 0.85 fold difference relative to the control sample, such as between a 0.2 and 0.7 fold difference relative to the control sample. In a further embodiment, the lower level is between a 0.25 and 0.75 fold difference relative to the control sample, such as those in accordance with the specific panel of analyte biomarkers according to the first aspect of the invention.

According to a further aspect of the invention, there is provided the use of IL-17, Cortisol (CORT), IL-6, Complement 3 (C3), Factor VII, Serum Amyloid P (SAP or APCS), Beta 2 Microglobulin, ICAM-1, IL-1 beta, TNF alpha, MIF, CD40, Cancer Antigen 125 (CA125), HCC 4 (CCL6; SCYA6), Eotaxin 3 (CCL26 or SCYA26), VEGF, Haptoglobin (HP), Apolipoprotein H (Beta-2 Glycoprotein), TIMP 1, ENA 78 (CXCL5), FABP (Fatty acid binding protein), MDC (CCL22), MIP 1 beta, PARC (p53-associated parkin-like cytoplasmic protein), Prostatic Acid Phosphatase, Stem Cell Factor (SCF), T3 Antibody, Thrombopoietin (THPO), Scl 70 Antibody, Histone H2B Antibody, Histone H1 Antibody, Histone Antibody, PM 1 Antibody, Histone H3 Antibody, Histone H2a Antibody, Anti Nuclear Antibody, SSB Antibody, Centromere Protein B Antibody, Rubeola, Hepatitis C Core, Hepatitis E Virus orf 3.3KD, Smith Antibody, HSP 32 HO Antibody, Parainfluenza 1, Hepatitis D, Proteinase 3 cANCA Antibody, HSP 71 Antibody, Collagen Type 2 Antibody, Mycoplasma pneumoniae (M. pneumoniae), Trypanosoma cruzi (T. cruzi), Hepatitis A, RNP Antibody, Hepatitis C NS4, RNP (a) Antibody, HIV 1 gp120, Chlamydia trachomatis (C. trachomatis), Helicobacter pylori (H. pylori), Mumps, Bordetella pertussis (B. pertussi), Beta-2 Glycoprotein Antibody (B2GP), Hepatitis C NS3, Collagen Type 4 Antibody (COL4), Poliovirus, Hepatitis C NS5, Ferritin (FTL), Fibrinogen (FGA), G-CSF, IL-12 p70, IL-15, IL-16, IL-18, IL-1 ra, IL-4, IL-7, IL-8, Leptin, MIP-1 alpha, PDGF (Platelet-derived growth factor), SOD, Ribosomal P Antibody, HSC 70 Antibody, HSP90 alpha Antibody, HSP90 beta Antibody and Varicella zoster (V. zoster; VZV) as a specific panel of analyte biomarkers for major depressive disorder, or predisposition thereto.

According to a further aspect of the invention, there is provided a method of diagnosing major depressive disorder, or predisposition thereto, in an individual thereto comprising
a) obtaining a biological sample from an individual;
b) quantifying the amounts of a panel of analyte biomarkers in the biological sample, wherein the panel of analyte biomarkers comprises IL-17, Cortisol (CORT), IL-6, Complement 3 (C3), Factor VII, Serum Amyloid P (SAP or APCS), Beta 2 Microglobulin, ICAM-1, IL-1 beta, TNF alpha, MIF, CD40, Cancer Antigen 125 (CA125), HCC 4 (CCL6; SCYA6), Eotaxin 3 (CCL26 or SCYA26), VEGF, Haptoglobin (HP), Apolipoprotein H (Beta-2 Glycoprotein), TIMP 1, ENA 78 (CXCL5), FABP (Fatty acid binding protein), MDC (CCL22), MIP 1 beta, PARC (p53-associated parkin-like cytoplasmic protein), Prostatic Acid Phosphatase, Stem Cell Factor (SCF), T3 Antibody, Thrombopoietin (THPO), Scl 70 Antibody, Histone H2B Antibody, Histone H1 Antibody, Histone Antibody, PM 1 Antibody, Histone H3 Antibody, Histone H2a Antibody, Anti Nuclear Antibody, SSB Antibody, Centromere Protein B Antibody, Rubeola, Hepatitis C Core, Hepatitis E Virus orf 3.3KD, Smith Antibody, HSP 32 HO Antibody, Parainfluenza 1, Hepatitis D, Proteinase 3 cANCA Antibody, HSP 71 Antibody, Collagen Type 2 Antibody, Mycoplasma pneumoniae (M. pneumoniae), Trypanosoma cruzi (T. cruzi), Hepatitis A, RNP Antibody, Hepatitis C NS4, RNP (a) Antibody, HIV 1 gp120, Chlamydia trachomatis (C. trachomatis), Helicobacter pylori (H. pylori), Mumps, Bordetella pertussis (B. pertussi), Beta-2 Glycoprotein Antibody (B2GP), Hepatitis C NS3, Collagen Type 4 Antibody (COL4), Poliovirus, Hepatitis C NS5, Ferritin (FTL), Fibrinogen (FGA), G-CSF, IL-12 p70, IL-15, IL-16, IL-18, IL-1 ra, IL-4, IL-7, IL-8, Leptin, MIP-1 alpha, PDGF (Platelet-derived growth factor), SOD, Ribosomal P Antibody, HSC 70 Antibody, HSP90 alpha Antibody, HSP90 beta Antibody and Varicella zoster (V. zoster; VZV); and
c) comparing the amounts of the panel of analyte biomarkers in the biological sample with the amounts present in a normal control biological sample from a normal subject, wherein a higher level of the panel of analyte biomarkers in the biological sample is indicative of major depressive disorder, or predisposition thereto.

In one embodiment, the higher level is a >1 fold difference relative to the control sample, such as a fold difference of 1.5, 2.0, 2.5, 3.0, 3.5, 4.0, 4.5, 5.0, 5.5, 6.0, 6.5, 7.0, 7.5, 8.0, 8.5, 9.0, 9.5, 10, 10.5, 11, 11.5, 12, 12.5, 15 or 20 or any ranges therebetween. In one embodiment, the higher level is between a 1 and 15 fold difference relative to the control sample, such as between a 1.5 and 12 fold difference relative to the control sample. In a further embodiment, the higher level is between a 1 and 7 fold difference relative to the control sample, such as those in accordance with the specific panel of analyte biomarkers according to the first aspect of the invention.

As used herein, the term “biosensor” means anything capable of detecting the presence of the biomarker. Examples of biosensors are described herein.

In one embodiment, one or more of the biomarkers defined hereinbefore may be io replaced by a molecule, or a measurable fragment of the molecule, found upstream or downstream of the biomarker in a biological pathway.

Biosensors according to the invention may comprise a ligand or ligands, as described herein, capable of specific binding to the peptide biomarker. Such biosensors are useful in detecting and/or quantifying a peptide of the invention.

Diagnostic kits for the diagnosis and monitoring of major depressive disorder are described herein. In one embodiment, the kits additionally contain a biosensor capable of detecting and/or quantifying a peptide biomarker.

Monitoring methods of the invention can be used to monitor onset, progression, stabilisation, amelioration and/or remission.

In methods of diagnosing or monitoring according to the invention, detecting and/or quantifying the peptide biomarker in a biological sample from a test subject may be performed on two or more occasions. Comparisons may be made between the level of biomarker in samples taken on two or more occasions. Assessment of any change in the level of the peptide biomarker in samples taken on two or more occasions may be performed. Modulation of the peptide biomarker level is useful as an indicator of the state of major depressive disorder or predisposition thereto. An increase in the level of the biomarker, over time is indicative of onset or progression, i.e. worsening of this disorder, whereas a decrease in the level of the peptide biomarker indicates amelioration or remission of the disorder, or vice versa.

A method of diagnosis of or monitoring according to the invention may comprise quantifying the peptide biomarker in a test biological sample from a test subject and comparing the level of the peptide present in said test sample with one or more controls.

The control used in a method of the invention can be one or more control(s) selected from the group consisting of: the level of biomarker peptide found in a normal control sample from a normal subject, a normal biomarker peptide level; a normal biomarker peptide range, the level in a sample from a subject with major depressive disorder, or a diagnosed predisposition thereto; major depressive disorder biomarker peptide level, or major depressive disorder biomarker peptide range.

In one embodiment, there is provided a method of diagnosing major depressive disorder, or predisposition thereto, which comprises:
(a) quantifying the amount of the peptide biomarker in a test biological sample; and
(b) comparing the amount of said peptide in said test sample with the amount present in a normal control biological sample from a normal subject.

For biomarkers which are increased in patients with major depressive disorder, a higher level of the peptide biomarker in the test sample relative to the level in the normal control is indicative of the presence of major depressive disorder, or predisposition thereto; an equivalent or lower level of the peptide in the test sample relative to the normal control is indicative of absence of major depressive disorder and/or absence of a predisposition thereto. For biomarkers which are decreased in patients with major depressive disorder, a lower level of the peptide biomarker in the test sample relative to the level in the normal control is indicative of the presence of major depressive disorder, or predisposition thereto; an equivalent or higher level of the peptide in the test sample relative to the normal control is indicative of absence of major depressive disorder and/or absence of a predisposition thereto.

The term “diagnosis” as used herein encompasses identification, confirmation, and/or characterisation of major depressive disorder, or predisposition thereto. By predisposition it is meant that a subject does not currently present with the disorder, but is liable to be affected by the disorder in time. Methods of monitoring and of diagnosis according to the invention are useful to confirm the existence of a disorder, or predisposition thereto; to monitor development of the disorder by assessing onset and progression, or to assess amelioration or regression of the disorder. Methods of monitoring and of diagnosis are also useful in methods for assessment of clinical screening, prognosis, choice of therapy, evaluation of therapeutic benefit, i.e. for drug screening and drug development.

Efficient diagnosis and monitoring methods provide very powerful “patient solutions” with the potential for improved prognosis, by establishing the correct diagnosis, allowing rapid identification of the most appropriate treatment (thus lessening unnecessary exposure to harmful drug side effects), reducing “down-time” and relapse rates.

Also provided is a method of monitoring efficacy of a therapy for major depressive disorder in a subject having such a disorder, suspected of having such a disorder, or of being predisposed thereto, comprising detecting and/or quantifying the peptide present in a biological sample from said subject. In monitoring methods, test samples may be taken on two or more occasions. The method may further comprise comparing the level of the biomarker(s) present in the test sample with one or more control(s) and/or with one or more previous test sample(s) taken earlier from the same test subject, e.g. prior to commencement of therapy, and/or from the same test subject at an earlier stage of therapy. The method may comprise detecting a change in the level of the biomarker(s) in test samples taken on different occasions.

The invention provides a method for monitoring efficacy of therapy for major depressive disorder in a subject, comprising:
(a) quantifying the amount of the peptide biomarker; and
(b) comparing the amount of said peptide in said test sample with the amount present in one or more control(s) and/or one or more previous test sample(s) taken at an earlier time from the same test subject.

For biomarkers which are increased in patients with major depressive disorder, a decrease in the level of the peptide biomarker in the test sample relative to the level in a previous test sample taken earlier from the same test subject is indicative of a beneficial effect, e.g. stabilisation or improvement, of said therapy on the disorder, suspected disorder or predisposition thereto. For biomarkers which are decreased in patients with major depressive disorder, an increase in the level of the peptide biomarker in the test sample relative to the level in a previous test sample taken earlier from the same test subject is indicative of a beneficial effect, e.g. stabilisation or improvement, of said therapy on the disorder, suspected disorder or predisposition thereto.

Methods for monitoring efficacy of a therapy can be used to monitor the therapeutic effectiveness of existing therapies and new therapies in human subjects and in non-human animals (e.g. in animal models). These monitoring methods can be incorporated into screens for new drug substances and combinations of substances.

Suitably, the time elapsed between taking samples from a subject undergoing diagnosis or monitoring will be 3 days, 5 days, a week, two weeks, a month, 2 months, 3 months, 6 or 12 months. Samples may be taken prior to and/or during and/or following an anti-depressant therapy. Samples can be taken at intervals over the remaining life, or a part thereof, of a subject.

The term “detecting” as used herein means confirming the presence of the peptide biomarker present in the sample. Quantifying the amount of the biomarker present in a sample may include determining the concentration of the peptide biomarker present in the sample. Detecting and/or quantifying may be performed directly on the sample, or indirectly on an extract therefrom, or on a dilution thereof.

In alternative aspects of the invention, the presence of the peptide biomarker is assessed by detecting and/or quantifying antibody or fragments thereof capable of specific binding to the biomarker that are generated by the subject's body in response to the peptide and thus are present in a biological sample from a subject having major depressive disorder or a predisposition thereto.

Detecting and/or quantifying can be performed by any method suitable to identify the presence and/or amount of a specific protein in a biological sample from a patient or a purification or extract of a biological sample or a dilution thereof. In methods of the invention, quantifying may be performed by measuring the concentration of the peptide biomarker in the sample or samples. Biological samples that may be tested in a method of the invention include cerebrospinal fluid (CSF), whole blood, blood serum, plasma, urine, saliva, or other bodily fluid (stool, tear fluid, synovial fluid, sputum), breath, e.g. as condensed breath, or an extract or purification therefrom, or dilution thereof. Biological samples also include tissue homogenates, tissue sections and biopsy specimens from a live subject, or taken post-mortem. The samples can be prepared, for example where appropriate diluted or concentrated, and stored in the usual manner.

Detection and/or quantification of peptide biomarkers may be performed by detection of the peptide biomarker or of a fragment thereof, e.g. a fragment with C-terminal truncation, or with N-terminal truncation. Fragments are suitably greater than 4 amino acids in length, for example 5, 6, 7, 8, 9, 10, 11, 12, 13, 14, 15, 16, 17, 18, 19, or 20 amino acids in length.

The biomarker may be directly detected, e.g. by SELDI or MALDI-TOF. Alternatively, the biomarker may be detected directly or indirectly via interaction with a ligand or ligands such as an antibody or a biomarker-binding fragment thereof, or other peptide, or ligand, e.g. aptamer, or oligonucleotide, capable of specifically binding the biomarker. The ligand may possess a detectable label, such as a luminescent, fluorescent or radioactive label, and/or an affinity tag.

For example, detecting and/or quantifying can be performed by one or more method(s) selected from the group consisting of: SELDI (-TOF), MALDI (-TOF), a 1-D gel-based analysis, a 2-D gel-based analysis, Mass spec (MS), reverse phase (RP) LC, size permeation (gel filtration), ion exchange, affinity, HPLC, UPLC and other LC or LC MS-based techniques. Appropriate LC MS techniques include ICAT® (Applied Biosystems, CA, USA), or iTRAQ® (Applied Biosystems, CA, USA). Liquid chromatography (e.g. high pressure liquid chromatography (HPLC) or low pressure liquid chromatography (LPLC)), thin-layer chromatography, NMR (nuclear magnetic resonance) spectroscopy could also be used.

Methods of diagnosing or monitoring according to the invention may comprise analysing a sample of cerebrospinal fluid (CSF) by SELDI TOF or MALDI TOF to detect the presence or level of the peptide biomarker. These methods are also suitable for clinical screening, prognosis, monitoring the results of therapy, identifying patients most likely to respond to a particular therapeutic treatment, for drug screening and development, and identification of new targets for drug treatment.

Detecting and/or quantifying the peptide biomarkers may be performed using an immunological method, involving an antibody, or a fragment thereof capable of specific binding to the peptide biomarker. Suitable immunological methods include sandwich immunoassays, such as sandwich ELISA, in which the detection of the peptide biomarkers is performed using two antibodies which recognize different epitopes on a peptide biomarker; radioimmunoassays (RIA), direct, indirect or competitive enzyme linked immunosorbent assays (ELISA), enzyme immunoassays (EIA), Fluorescence immunoassays (FIA), western blotting, immunoprecipitation and any particle-based immunoassay (e.g. using gold, silver, or latex particles, magnetic particles, or Q-dots). Immunological methods may be performed, for example, in microtitre plate or strip format.

Immunological methods in accordance with the invention may be based, for example, on any of the following methods.

Immunoprecipitation is the simplest immunoassay method; this measures the quantity of precipitate, which forms after the reagent antibody has incubated with the sample and reacted with the target antigen present therein to form an insoluble aggregate. Immunoprecipitation reactions may be qualitative or quantitative.

In particle immunoassays, several antibodies are linked to the particle, and the particle is able to bind many antigen molecules simultaneously. This greatly accelerates the speed of the visible reaction. This allows rapid and sensitive detection of the biomarker.

In immunonephelometry, the interaction of an antibody and target antigen on the biomarker results in the formation of immune complexes that are too small to precipitate. However, these complexes will scatter incident light and this can be measured using a nephelometer. The antigen, i.e. biomarker, concentration can be determined within minutes of the reaction.

Radioimmunoassay (RIA) methods employ radioactive isotopes such as 1¹²⁵ to label either the antigen or antibody. The isotope used emits gamma rays, which are usually measured following removal of unbound (free) radiolabel. The major advantages of RIA, compared with other immunoassays, are higher sensitivity, easy signal detection, and well-established, rapid assays. The major disadvantages are the health and safety risks posed by the use of radiation and the time and expense associated with maintaining a licensed radiation safety and disposal program. For this reason, RIA has been largely replaced in routine clinical laboratory practice by enzyme immunoassays.

Enzyme (EIA) immunoassays were developed as an alternative to radioimmunoassays (RIA). These methods use an enzyme to label either the antibody or target antigen. The sensitivity of EIA approaches that for RIA, without the danger posed by radioactive isotopes. One of the most widely used EIA methods for detection is the enzyme-linked immunosorbent assay (ELISA). ELISA methods may use two antibodies one of which is specific for the target antigen and the other of which is coupled to an enzyme, addition of the substrate for the enzyme results in production of a chemiluminescent or fluorescent signal.

Fluorescent immunoassay (FIA) refers to immunoassays which utilize a fluorescent label or an enzyme label which acts on the substrate to form a fluorescent product. Fluorescent measurements are inherently more sensitive than colorimetric (spectrophotometric) measurements. Therefore, FIA methods have greater analytical sensitivity than EIA methods, which employ absorbance (optical density) measurement.

Chemiluminescent immunoassays utilize a chemiluminescent label, which produces light when excited by chemical energy; the emissions are measured using a light detector.

Immunological methods according to the invention can thus be performed using well-known methods. Any direct (e.g., using a sensor chip) or indirect procedure may be used in the detection of peptide biomarkers of the invention.

The Biotin-Avidin or Biotin-Streptavidin systems are generic labelling systems that can be adapted for use in immunological methods of the invention. One binding partner (hapten, antigen, ligand, aptamer, antibody, enzyme etc) is labelled with biotin and the other partner (surface, e.g. well, bead, sensor etc) is labelled with avidin or streptavidin. This is conventional technology for immunoassays, gene probe assays and (bio)sensors, but is an indirect immobilisation route rather than a direct one. For example a biotinylated ligand (e.g. antibody or aptamer) specific for a peptide biomarker of the invention may be immobilised on an avidin or streptavidin surface, the immobilised ligand may then be exposed to a sample containing or suspected of containing the peptide biomarker in order to detect and/or quantify a peptide biomarker of the invention. Detection and/or quantification of the immobilised antigen may then be performed by an immunological method as described herein.

The term “antibody” as used herein includes, but is not limited to: polyclonal, monoclonal, bispecific, humanised or chimeric antibodies, single chain antibodies, Fab fragments and F(ab′)₂ fragments, fragments produced by a Fab expression library, anti-idiotypic (anti-Id) antibodies and epitope-binding fragments of any of the above. The term “antibody” as used herein also refers to immunoglobulin molecules and immunologically-active portions of immunoglobulin molecules, i.e., molecules that contain an antigen binding site that specifically binds an antigen. The immunoglobulin molecules of the invention can be of any class (e. g., IgG, IgE, IgM, IgD and IgA) or subclass of immunoglobulin molecule.

The identification of key biomarkers specific to a disease is central to integration of diagnostic procedures and therapeutic regimes. Using predictive biomarkers appropriate diagnostic tools such as biosensors can be developed, accordingly, in methods and uses of the invention, detecting and quantifying can be performed using a biosensor, microanalytical system, microengineered system, microseparation system, immunochromatography system or other suitable analytical devices. The biosensor may incorporate an immunological method for detection of the biomarker(s), electrical, thermal, magnetic, optical (e.g. hologram) or acoustic technologies. Using such biosensors, it is possible to detect the target biomarker(s) at the anticipated concentrations found in biological samples.

Thus, according to a further aspect of the invention there is provided an apparatus for diagnosing or monitoring major depressive disorder which comprises a biosensor, microanalytical, microengineered, microseparation and/or immunochromatography system configured to detect and/or quantify any of the biomarkers defined herein.

The biomarker(s) of the invention can be detected using a biosensor incorporating technologies based on “smart” holograms, or high frequency acoustic systems, such systems are particularly amenable to “bar code” or array configurations.

In smart hologram sensors (Smart Holograms Ltd, Cambridge, UK), a holographic image is stored in a thin polymer film that is sensitised to react specifically with the biomarker. On exposure, the biomarker reacts with the polymer leading to an alteration in the image displayed by the hologram. The test result read-out can be a change in the optical brightness, image, colour and/or position of the image. For qualitative and semi-quantitative applications, a sensor hologram can be read by eye, thus removing the need for detection equipment. A simple colour sensor can be used to read the signal when quantitative measurements are required. Opacity or colour of the sample does not interfere with operation of the sensor. The format of the sensor allows multiplexing for simultaneous detection of several substances. Reversible and irreversible sensors can be designed to meet different requirements, and continuous monitoring of a particular biomarker of interest is feasible.

Suitably, biosensors for detection of one or more biomarkers of the invention combine biomolecular recognition with appropriate means to convert detection of the presence, or quantitation, of the biomarker in the sample into a signal. Biosensors can be adapted for “alternate site” diagnostic testing, e.g. in the ward, outpatients' department, surgery, home, field and workplace.

Biosensors to detect one or more biomarkers of the invention include acoustic, plasmon resonance, holographic and microengineered sensors. Imprinted recognition elements, thin film transistor technology, magnetic acoustic resonator devices and other novel acousto-electrical systems may be employed in biosensors for detection of the one or more biomarkers of the invention.

Methods involving detection and/or quantification of one or more peptide biomarkers of the invention can be performed on bench-top instruments, or can be incorporated onto disposable, diagnostic or monitoring platforms that can be used in a non-laboratory environment, e.g. in the physician's office or at the patient's bedside. Suitable biosensors for performing methods of the invention include “credit” cards with optical or acoustic readers. Biosensors can be configured to allow the data collected to be electronically transmitted to the physician for interpretation and thus can form the basis for e-neuromedicine.

Any suitable animal may be used as a subject non-human animal, for example a non-human primate, horse, cow, pig, goat, sheep, dog, cat, fish, rodent, e.g. guinea pig, rat or mouse; insect (e.g. Drosophila), amphibian (e.g. Xenopus) or C. elegans.

The test substance can be a known chemical or pharmaceutical substance, such as, but not limited to, an anti-depressive disorder therapeutic; or the test substance can be novel synthetic or natural chemical entity, or a combination of two or more of the aforesaid substances.

There is provided a method of identifying a substance capable of promoting or suppressing the generation of the peptide biomarker in a subject, comprising exposing a test cell to a test substance and monitoring the level of the peptide biomarker within said test cell, or secreted by said test cell.

The test cell could be prokaryotic, however a eukaryotic cell will suitably be employed in cell-based testing methods. Suitably, the eukaryotic cell is a yeast cell, insect cell, Drosophila cell, amphibian cell (e.g. from Xenopus), C. elegans cell or is a cell of human, non-human primate, equine, bovine, porcine, caprine, ovine, canine, feline, piscine, rodent or murine origin.

In methods for identifying substances of potential therapeutic use, non-human animals or cells can be used that are capable of expressing the peptide.

Screening methods also encompass a method of identifying a ligand capable of binding to the peptide biomarker according to the invention, comprising incubating a test substance in the presence of the peptide biomarker in conditions appropriate for binding, and detecting and/or quantifying binding of the peptide to said test substance.

High-throughput screening technologies based on the biomarker, uses and methods of the invention, e.g. configured in an array format, are suitable to monitor biomarker signatures for the identification of potentially useful therapeutic compounds, e.g. ligands such as natural compounds, synthetic chemical compounds (e.g. from combinatorial libraries), peptides, monoclonal or polyclonal antibodies or fragments thereof, which may be capable of binding the biomarker.

Methods of the invention can be performed in array format, e.g. on a chip, or as a multiwell array. Methods can be adapted into platforms for single tests, or multiple identical or multiple non-identical tests, and can be performed in high throughput format. Methods of the invention may comprise performing one or more additional, different tests to confirm or exclude diagnosis, and/or to further characterise a condition.

The invention further provides a substance, e.g. a ligand, identified or identifiable by an identification or screening method or use of the invention. Such substances may be capable of inhibiting, directly or indirectly, the activity of the peptide biomarker, or of suppressing generation of the peptide biomarker. The term “substances” includes substances that do not directly bind the peptide biomarker and directly modulate a function, but instead indirectly modulate a function of the peptide biomarker. Ligands are also included in the term substances; ligands of the invention (e.g. a natural or synthetic chemical compound, peptide, aptamer, oligonucleotide, antibody or antibody fragment) are capable of binding, suitably specific binding, to the peptide.

The invention further provides a substance according to the invention for use in the treatment of major depressive disorder, or predisposition thereto.

Also provided is the use of a substance according to the invention in the treatment of major depressive disorder, or predisposition thereto.

Also provided is the use of a substance according to the invention as a medicament.

A kit for diagnosing or monitoring major depressive disorder, or predisposition thereto is provided. Suitably a kit according to the invention may contain one or more components selected from the group: a ligand specific for the peptide biomarker or a structural/shape mimic of the peptide biomarker, one or more controls, one or more reagents and one or more consumables; optionally together with instructions for use of the kit in accordance with any of the methods defined herein.

The identification of biomarkers for major depressive disorder permits integration of diagnostic procedures and therapeutic regimes. Currently there are significant delays in determining effective treatment and hitherto it has not been possible to perform rapid assessment of drug response. Traditionally, many anti-depressant therapies have required treatment trials lasting weeks to months for a given therapeutic approach. Detection of a peptide biomarker of the invention can be used to screen subjects prior to their participation in clinical trials. The biomarkers provide the means to indicate therapeutic response, failure to respond, unfavourable side-effect profile, degree of medication compliance and achievement of adequate serum drug levels. The biomarkers may be used to provide warning of adverse drug response. Biomarkers are useful in development of personalized brain therapies, as assessment of response can be used to fine-tune dosage, minimise the number of prescribed medications, reduce the delay in attaining effective therapy and avoid adverse drug reactions. Thus by monitoring a biomarker of the invention, patient care can be tailored precisely to match the needs determined by the disorder and the pharmacogenomic profile of the patient, the biomarker can thus be used to titrate the optimal dose, predict a positive therapeutic response and identify those patients at high risk of severe side effects.

Biomarker-based tests provide a first line assessment of ‘new’ patients, and provide objective measures for accurate and rapid diagnosis, in a time frame and with precision, not achievable using the current subjective measures.

Furthermore, diagnostic biomarker tests are useful to identify family members or patients at high risk of developing major depressive disorder. This permits initiation of appropriate therapy, or preventive measures, e.g. managing risk factors. These approaches are recognised to improve outcome and may prevent overt onset of the disorder.

Biomarker monitoring methods, biosensors and kits are also vital as patient monitoring tools, to enable the physician to determine whether relapse is due to worsening of the disorder, poor patient compliance or substance abuse. If pharmacological treatment is assessed to be inadequate, then therapy can be reinstated or increased; a change in therapy can be given if appropriate. As the biomarkers are sensitive to the state of the disorder, they provide an indication of the impact of drug therapy or of substance abuse.

The following studies illustrate the invention.

### Study 1

Study 1 measured levels of 247 molecules in serum collected from 50 major depressive disorder (MDD) patients and 50 well matched controls. Levels of all molecular analytes were determined using a highly reproducible multiplexed immunoassay platform. The correlation structure between all analytes was assessed to infer potential co-regulation structures.

A panel of 97 markers was found to be significantly altered in the MDD group. This panel of markers was found to yield a sensitivity of 92% and a specificity of 98%. These abnormalities remained significant after adjustment for all recorded baseline characteristics including age, sex, body mass index and smoking. Among the significant markers, a highly prominent correlation structure was found.

### Methodology

### Patients

In the present study, samples were investigated from patients suffering from major depressive disorder (MDD) (n=50) and well matched controls (n=50). All individuals were fasted at the time of blood sample collection and featured no co-morbidities. The ethical committees of the medical faculties of the partner universities approved the protocols of this study. Informed consent was given in writing by all participants and clinical investigations were conducted according to the principles expressed in the Declaration of Helsinki.

### Sample Preparation

Blood was collected in S-Monovette 7.5 mL serum tubes (Sarstedt), incubated at room temperature for 2 hours to allow for blood coagulation and then centrifuged at 4000×g for 5 minutes. The supernatant was stored at −80° C. in Low Binding Eppendorf tubes.

### Assay Methods

A total of 247 analytes were measured using a set of proprietary multiplexed immunoassays (Human MAP; Supplementary Table S1) at Rules Based Medicine in their Luminex-based, CLIA-certified laboratory (however measurement could equally be performed using singleton ELISA). Each antigen assay was calibrated using 8-point standard curves conducted in duplicate, and raw intensity measurements were interpreted into final protein concentrations. Machine performance was verified using quality control samples at low, medium, and high levels for each analyte in duplicate. All standard and quality control samples were in a complex plasma-based matrix to match the sample background. The autoimmune and infectious disease assays were qualitative and the results obtained for unknown samples were compared with established cut-off values. Because sera were analyzed at a previously optimized dilution, any sample exceeding the maximum concentration of the calibration curve was arbitrarily assigned the concentration of the highest standard, whereas those assayed below the minimum concentration of the calibration curve were assigned the value 0.0. For analysis, samples were ordered in a manner to avoid any sequential bias due to the presence or absence of disease, patient age, or age of serum sample. Generally, samples alternated between cases and controls.

### Statistical Analysis

The distribution of the data was examined using standard statistics to assess the necessity for transformations, the presence of outliers or artefactual findings. Parametric (T-test) and non-parametric (Wilcoxon Rank Sum statistics) univariate methods were applied to identify significant differences of molecular levels between the disease and control groups. A p-value of less than 0.05 was considered as being significant. The False Discovery Rate (FDR) was controlled according to Benjamini et al. (J Roy Statist Soc Ser B. 1995; 57:289-300). Multivariate statistics (Principal Component Analysis, PCA and Partial Least Squares Discriminant Analysis, PLS-DA) were applied to identify potential groups of markers that discriminated patient from control groups and to assess the agreement with univariate methods.

The effect of the baseline characteristics on the markers was accounted for using ANCOVA models. Adjustments were made for the effects of age, sex, body mass index, smoking, cannabis and the date of blood sample collection.

### Results

This study investigated levels of 247 molecular analytes in serum from 50 patients suffering from major depressive disorder and well matched controls (n=50). Demographic details can be found in Table 2:

TABLE 2
Demographic details of patients and healthy volunteers

| | Healthy Controls | Major Depressive |
|---|---|---|
| | (MDD) | Disorder |
| Number | 50 | 50 |
| Sex (m/f) | 16/34 | 17/32 + 1 |
| Age | 45 · 9 ± 9 · 5 | 46 · 1 ± 13 · 4 |

Applying T-tests, levels of 97 analytes were found to be significantly altered between the disease and the control group (Table 3). Adjustment for multiple comparisons yielded q-values ranging from 0 to 0.13. These values were in very good agreement with the results obtained from non-parametric and multivariate analyses.

TABLE 3
Summary of significant findings

| | | | Fold |
|---|---|---|---|
| Analyte | P-value | Q-value | change |
| IL-15 | 1.84E−20 | 4.43E−18 | 1.810192 |
| Scl 70 Antibody | 4.54E−12 | 5.47E−10 | 2.527628 |
| Histone Antibody | 1.16E−11 | 9.33E−10 | 1.834878 |
| IL-7 | 5.43E−11 | 3.27E−09 | 1.351796 |
| Histone H2b Antibody | 1.12E−10 | 5.40E−09 | 1.721451 |
| Histone H1 Antibody | 3.80E−10 | 1.53E−08 | 3.046246 |
| IL-5 | 1.41E−09 | 4.84E−08 | 0.427325 |
| PM 1 Antibody | 1.04E−07 | 3.12E−06 | 1.301572 |
| MDC | 2.44E−07 | 6.54E−06 | 1.358487 |
| IL-17 | 4.05E−07 | 9.77E−06 | 1.432133 |
| Histone H3 Antibody | 4.82E−07 | 1.06E−05 | 2.743889 |
| IgA | 3.11E−06 | 6.25E−05 | 0.691262 |
| Cortisol | 4.86E−06 | 9.02E−05 | 1.462218 |
| Fibrinogen | 7.40E−06 | 0.000127 | 9.923319 |
| MIP-1 alpha | 9.08E−06 | 0.000146 | 1.346904 |
| IL-12 p70 | 1.18E−05 | 0.000178 | 1.178722 |
| Ferritin | 1.26E−05 | 0.000179 | 2.749356 |
| Apolipoprotein A1 | 1.36E−05 | 0.000182 | 0.720232 |
| Anti Nuclear Antibody | 1.61E−05 | 0.000204 | 1.385441 |
| Ribosomal P Antibody | 2.70E−05 | 0.000326 | 1.216282 |
| IL-6 | 3.65E−05 | 0.000419 | 6.652215 |
| IL-1ra | 4.06E−05 | 0.000445 | 1.963259 |
| SSB Antibody | 8.71E−05 | 0.000913 | 1.213557 |
| Centromere Protein B Antibody | 9.23E−05 | 0.000927 | 1.338174 |
| Complement 3 | 0.000101 | 0.000978 | 1.148825 |
| Factor VII | 0.000119 | 0.001065 | 1.259236 |
| PARC | 0.000119 | 0.001065 | 1.368787 |
| MIP-1 beta | 0.000172 | 0.001479 | 1.306585 |
| T3 Antibody | 0.000221 | 0.001822 | 1.325668 |
| IL-8 | 0.000227 | 0.001822 | 1.74579 |
| Serum Amyloid P | 0.000267 | 0.002078 | 1.254653 |
| Beta 2 Microglobulin | 0.00032 | 0.002413 | 1.156625 |
| Rubeola | 0.000384 | 0.002806 | 1.366355 |
| Hepatitis C Core | 0.000509 | 0.003609 | 1.384339 |
| HSC 70 Antibody | 0.000636 | 0.004266 | 1.20807 |
| ICAM-1 | 0.000637 | 0.004266 | 1.180603 |
| IL-1 beta | 0.00074 | 0.004823 | 1.378796 |
| G-CSF | 0.000793 | 0.005028 | 1.414499 |
| IL-16 | 0.000843 | 0.005212 | 1.309694 |
| TNF alpha | 0.000987 | 0.005892 | 1.246216 |
| Hepatitis E Virus orf 3.3KD | 0.001002 | 0.005892 | 1.322729 |
| HSP 90 alpha Antibody | 0.001403 | 0.008048 | 1.623888 |
| Smith Antibody | 0.001584 | 0.008789 | 1.17663 |
| HSP32 HO Antibody | 0.001605 | 0.008789 | 1.22986 |
| Parainfluenza 1 | 0.00178 | 0.009531 | 1.50474 |
| IL-18 | 0.002148 | 0.011256 | 1.333671 |
| TSP 1 | 0.002438 | 0.012499 | 0.878328 |
| Peptide YY | 0.002659 | 0.013137 | 0.069199 |
| Thrombopoietin | 0.002671 | 0.013137 | 1.132172 |
| HSP90 beta Antibody | 0.003067 | 0.014782 | 1.608074 |
| Hepatitis D | 0.003447 | 0.016291 | 1.437876 |
| Creatine Kinase MB | 0.004325 | 0.019858 | 0.728165 |
| MIF | 0.004367 | 0.019858 | 3.131054 |
| Proteinase 3 cANCA Antibody | 0.004882 | 0.021789 | 1.209793 |
| Angiotensinogen | 0.004976 | 0.021804 | 0.29868 |
| NrCAM | 0.005174 | 0.021904 | 0.654855 |
| CD40 | 0.005181 | 0.021904 | 1.150131 |
| Sortilin | 0.00657 | 0.027298 | 0.839157 |
| HSP 71 Antibody | 0.006924 | 0.028283 | 1.217916 |
| Collagen Type 2 Antibody | 0.007155 | 0.028738 | 1.620513 |
| M. pneumoniae | 0.007564 | 0.029885 | 1.461853 |
| T. cruzi | 0.007991 | 0.031063 | 1.223977 |
| Cancer Antigen 125 | 0.008893 | 0.03402 | 1.549108 |
| Hepatitis A | 0.009614 | 0.036202 | 1.376552 |
| RNP Antibody | 0.009979 | 0.036999 | 1.15717 |
| V. zoster | 0.010857 | 0.039646 | 1.467239 |
| ENA 78 | 0.011328 | 0.040747 | 1.321957 |
| HCC 4 | 0.012232 | 0.043352 | 1.253832 |
| Leptin | 0.013438 | 0.04674 | 1.996765 |
| Eotaxin 3 | 0.013576 | 0.04674 | 3.395143 |
| Hepatitis C NS4 | 0.014102 | 0.047254 | 1.353607 |
| VEGF | 0.014117 | 0.047254 | 1.203276 |
| IL-4 | 0.015194 | 0.050151 | 1.219923 |
| Endothelin 1 | 0.015399 | 0.050151 | 0.535709 |
| RNP a Antibody | 0.015997 | 0.051404 | 1.97955 |
| Haptoglobin | 0.016727 | 0.053041 | 1.369183 |
| HIV-1 gp120 | 0.017558 | 0.054954 | 1.358895 |
| C. trachomatis | 0.018355 | 0.056711 | 1.433513 |
| SOD | 0.020178 | 0.061556 | 1.535377 |
| IL-1 alpha | 0.020632 | 0.062155 | 0.396971 |
| H. pylori | 0.021665 | 0.06446 | 2.879708 |
| IL-13 | 0.023992 | 0.069933 | 0.819648 |
| Mumps | 0.024085 | 0.069933 | 1.389947 |
| B. pertussis | 0.029454 | 0.084504 | 1.450379 |
| PDGF | 0.034487 | 0.09778 | 1.168285 |
| Prostatic Acid Phosphatase | 0.035522 | 0.099544 | 1.148206 |
| FABP | 0.037437 | 0.103705 | 1.465374 |
| Apolipoprotein H | 0.039494 | 0.107575 | 1.103694 |
| Beta 2 Glycoprotein Antibody | 0.039727 | 0.107575 | 1.341369 |
| CTGF Connective Tissue Growth | 0.041504 | 0.109816 | 0.858914 |
| Factor. | | | |
| Stem Cell Factor | 0.041921 | 0.109816 | 1.116618 |
| Hepatitis C NS3 | 0.041922 | 0.109816 | 1.198521 |
| Collagen Type 4 Antibody | 0.045288 | 0.117359 | 1.087214 |
| Polio Virus | 0.047526 | 0.121847 | 1.139923 |
| Histone H2a Antibody | 0.048145 | 0.122135 | 1.292166 |
| TIMP 1 | 0.049507 | 0.124284 | 1.052848 |
| Hepatitis C NS5 | 0.052793 | 0.131166 | 1.167106 |

### Study 2

Study 2 was performed in an analogous manner to Study 1. This study investigated levels of 247 molecular analytes in serum from 35 patients suffering from first episode major depressive disorder and well matched controls (n=40).

The patient group were acutely ill, antipsychotic-naïve (n=22) or had been off medication for at least six weeks prior to sample collection (n=13). All cohorts were matched for age and gender and only subjects with no medical co-morbidities or substance abuse were included. Demographic details can be found in Table 4:

TABLE 4
Demographic details of patients and healthy volunteers

| | Healthy Controls | Major Depressive |
|---|---|---|
| | (MDD) | Disorder |
| Number | 40 | 35 |
| Sex (m/f) | 26/14 | 13/22 |
| Age | 36 ± 11 | 40 ± 14 |

Applying T-tests, levels of 8 analytes were found to be significantly altered between the disease and the control group (Table 5).

TABLE 5
Summary of significant findings

| Analyte | P-value |
|---|---|
| Alpha-Fetoprotein | 0.001 |
| SOD | 0.004 |
| Glutathione S-Transferase-α | 0.015 |
| IL-15 | 0.014 |
| Eotaxin | 0.021 |
| Toxoplasma | 0.028 |
| IGF-BP2 | 0.04 |
| Brain-Derived Neurotrophic Factor | 0.046 |

1. Use of IL-17, IgA, Cortisol (CORT), Apolipoprotein A1, IL-6, Complement 3 (C3), Factor VII, Serum Amyloid P (SAP or APCS), Beta 2 Microglobulin, ICAM-1, IL-1 beta, TNF alpha, MIF, Angiotensinogen, NrCAM (Neuronal cell adhesion molecule), CD40, Cancer Antigen 125 (CA125), HCC 4 (CCL6; SCYA6), Eotaxin 3 (CCL26 or SCYA26), VEGF, Haptoglobin (HP), IL-1 alpha, Apolipoprotein H (Beta-2 Glycoprotein) and TIMP 1 as a specific panel of analyte biomarkers for major depressive disorder, or predisposition thereto.

2. Use as defined in claim 1, wherein the panel additionally comprises one or more analyte biomarkers selected from: Creatine Kinase MB (CK-MB), ENA 78 (CXCL5), Endothelin 1, FABP (Fatty acid binding protein), MDC (CCL22), MIP 1 beta, PARC (p53-associated parkin-like cytoplasmic protein), Peptide YY (PYY), Prostatic Acid Phosphatase, Sortilin (SORT), Stem Cell Factor (SCF), T3 Antibody, Thrombopoietin (THPO), TSP 1 (thrombospondin-1), Scl 70 Antibody, Histone H2B Antibody, Histone H1 Antibody, Histone Antibody, PM 1 Antibody, Histone H3 Antibody, Histone H2a Antibody, Anti Nuclear Antibody, SSB Antibody, Centromere Protein B Antibody, Rubeola, Hepatitis C Core, Hepatitis E Virus orf 3.3KD, Smith Antibody, HSP 32 HO Antibody, Parainfluenza 1, Hepatitis D, Proteinase 3 cANCA Antibody, HSP 71 Antibody, Collagen Type 2 Antibody, Mycoplasma pneumoniae (M. pneumoniae), Trypanosoma cruzi (T. cruzi), Hepatitis A, RNP Antibody, Hepatitis C NS4, RNP (a) Antibody, HIV 1 gp120, Chlamydia trachomatis (C. trachomatis), Helicobacter pylori (H. pylori), Mumps, Bordetella pertussis (B. pertussi), Beta-2 Glycoprotein Antibody (B2GP), Hepatitis C NS3, Collagen Type 4 Antibody (COL4), Poliovirus, Hepatitis C NS5, CTGF (Connective Tissue Growth Factor), Ferritin (FTL), Fibrinogen (FGA), GCSF, IL-12 p70, IL-13, IL-15, IL-16, IL-18, IL-1 ra, IL-4, IL-5, IL-7, IL-8, Leptin, MIP-1 alpha, PDGF (Platelet-derived growth factor), SOD, Ribosomal P Antibody, HSC 70 Antibody, HSP90 alpha Antibody, HSP90 beta Antibody and Varicella zoster (V zoster; VZV).

3. Use as defined in claim 1 or claim 2, wherein the panel additionally comprises one or more analyte biomarkers selected from: Alpha-Fetoprotein, Glutathione S-Transferase-α, Eotaxin, Toxoplasma, IGF-BP2 and Brain-Derived Neurotrophic Factor.

4. (canceled)

5. (canceled)

6. Use as defined in claim 2, wherein the one or more analyte biomarkers are selected from Alpha-Fetoprotein, Bordetella pertussis (B. pertussi), Hepatitis C NS5 and Beta-2 Glycoprotein Antibody (B2GP).

7. (canceled)

8. Use as defined in claim 1, wherein one or more of the biomarkers may be replaced by a molecule, or a measurable fragment of the molecule, found upstream or downstream of the biomarker in a biological pathway.

9. A method of diagnosing major depressive disorder, or predisposition in an individual thereto, comprising:
obtaining a biological sample from an individual;
quantifying the amounts of the analyte biomarkers as defined in claim 1;
comparing the amounts of the analyte biomarkers in the biological sample with the amounts present in a normal control biological sample from a normal subject, such that a difference in the level of the analyte biomarkers in the biological sample is indicative of major depressive disorder, or predisposition thereto.

10. A method of monitoring efficacy of a therapy in a subject having, suspected of having, or of being predisposed to major depressive disorder, comprising detecting and/or quantifying, in a sample from said subject, the analyte biomarkers as defined in claim 1.

11. A method as defined in claim 9, which is conducted on samples taken on two or more occasions from a test subject.

12. A method as defined in claim 11, further comprising comparing the level of the biomarker present in samples taken on two or more occasions.

13. A method as defined in claim 9, comprising comparing the amount of the biomarker in said test sample with the amount present in one or more samples taken from said subject prior to commencement of therapy, and/or one or more samples taken from said subject at an earlier stage of therapy.

14. A method as defined in claim 10, further comprising detecting a change in the amount of the biomarker in samples taken on two or more occasions.

15. A method as defined in claim 9, comprising comparing the amount of the biomarker present in said test sample with one or more controls.

16. A method as defined in claim 15, comprising comparing the amount of the biomarker in a test sample with the amount of the biomarker present in a sample from a normal subject.

17. A method as defined in claim 9, wherein samples are taken prior to and/or during and/or following therapy for major depressive disorder.

18. A method as defined in claim 10, wherein samples are taken at intervals over the remaining life, or a part thereof, of a subject.

19. A method as defined in claim 9, wherein quantifying is performed by measuring the concentration of the analyte biomarker in the sample.

20. A method as defined in any claim 10, wherein detecting and/or quantifying is performed by one or more methods selected from SELDI (-TOF), MALDI (-TOF), a 1-D gel-based analysis, a 2-D gel-based analysis, Mass spec (MS), reverse phase (RP) LC, size permeation (gel filtration), ion exchange, affinity, HPLC, UPLC or other LC or LC-MS-based technique.

21. A method as defined in claim 10, wherein detecting and/or quantifying is performed using an immunological method.

22. A method as defined in claim 10, wherein the detecting and/or quantifying is performed using a biosensor or a microanalytical, microengineered, microseparation or immunochromatography system.

23. A method as defined in claim 9, wherein the biological sample is cerebrospinal fluid, whole blood, blood serum, plasma, urine, saliva, or other bodily fluid, or breath, condensed breath, or an extract or purification therefrom, or dilution thereof.

24. (canceled)
